(19) United States
(12) Patent Application Publication
(10) Pub. No.: US 2015/0320424 A1
(43) Pub. Date: Nov. 12, 2015

(54) Self-Expanding Anastomosis Device

(71) Applicant: UNIVERSITY OF STRATHCLYDE, Glasgow (GB)

(72) Inventor: Terrence GOURLAY, Glasgow (GB)

(21) Appl. No.: 14/651,482

(22) PCT Filed: Dec. 10, 2013
(86) PCT No.: PCT/GB2013/053249

§ 371 Date: Jun. 11, 2015

(30) Foreign Application Priority Data
Dec. 12, 2012 (GB) 1222340.0

(51) Int. Cl. A61B 17/11; A61B 5/026; A61B 5/021

(52) U.S. Cl. CPC A61B 17/11; A61B 5/021; A61B 5/026; A61B 2017/1107; A61B 2017/00022

(57) ABSTRACT

A size-variable Implantable device having a body including at least one attaching portion configured for securing the device to a surgical site of a patient; a size-controlling member configured for controlling at least one dimension of the device; and an actuator arranged to actuate the size-controlling member. The device may include a mounting portion for mounting one or more expandable conduits. The at least one attaching portion may be made from a deformable, e.g. an expandable and/or contractible, material, which may allow expansion and/or contraction of the size-variable implantable device. The device may include one or more sensors for detecting, measuring, collecting, storing, and/or communicating data in the region of deployment of the device, which may cause the actuator to actuate the size-controlling device when one or more of the detected parameters reach a predetermined value.

### FIELD OF INVENTION

The present invention relates to a size-variable implantable device, and in particular, though not exclusively, to a self-expandable implantable device configured for securing the device to a surgical site of a patient.

### BACKGROUND TO INVENTION

One in every 145 children in the United Kingdom is born with a congenital heart defect (CHD). One of the commonest malformations involves the vascular pathway from the right ventricle to the king circulation. This can be very poorly developed or completely absent, with blood entering the pulmonary circulation via an alternative route. Conditions in which this occurs include Tetrology of Fallot, Truncus Arteriosus, Pulmonary atresia. Another situation of absent Right Ventricle (RV) to Pulmonary Artery (PA) connection is surgically induced with the Ross procedure—in this operation the patient's diseased aortic valve is replaced by the patients own pulmonary valve, which they requires replacement.

Some types of complex cardiac lesions including CHDs can be corrected through cardiothoracic procedures such as the implantation of an artificial blood vessel, known as a graft or a conduit, to create an artificial pathway, e.g. from the right ventricle to the pulmonary circulation. These procedures require open heart surgery, which is invasive and life challenging.

A further technical challenge is created by the physical growth of children from infancy to adolescence. As the child grows, the adequate size of the conduit required to perform its function changes. Under current procedures, as the child grows, he/she needs to undergo a number of life-threatening procedures to introduce larger conduits. The number of revision procedures is typically two or three, in some cases up to four or five times. Revision surgery carries with it an increased risk of mortality to the patient, is extremely costly in terms of direct care, and impacts negatively on quality of life.

Current procedures are based on two separate approaches, each of which carrying a number of disadvantages.

A first approach involves the use of artificial prosthetic conduits that are made of a durable material. Although these conduits offer the benefit of durability, they do not grow with the patient and therefore need multiple replacements. Each revision carries an increased mortality risk; at the fourth revision, average mortality is over 15%.

A second approach involves the use of homographic conduits. These offer improved mechanical properties, but tend to degrade and/or calcify. In addition, they do not grow with the patient and need multiple replacements.

There has been extensive research into expandable conduits, and balloon-expandable conduits are known. However, these expandable conduits do not affect the size of the anastomosis site. As the anastomosis site remains the same size, this can creates a stenosis when the conduit is expanded. Thus, the use of balloon-expandable conduits does not eliminate the need for future revision surgery during child growth.

Other physiological conditions or pathologies may also benefit from the use of size-variable implantable devices, and in particular, of contractible implantable devices. For example, gastric reflux disease can be caused by abnormal relaxation of the lower oesophageal sphincter. Another example includes patients suffering from abnormal digestive systems, caused by abnormal relaxation of the anal sphincter.

International application Publication No. WO 2006/105084 (Cartledge et al.) discloses an implantable device for controlling the internal circumference of an anatomical orifice or lumen. The device comprises a circular implant body which is provided with adjustable corrugated sections alternating with grommet-like attachment means having narrowed intermediate neck portions. The corrugated sections fold and unfold as the circumference of the implant body shortens or lengthens. However, the provision of corrugations may cause damage and/or infection to a patient's tissue by trapping tissues between the corrugations. Such a device is also not suitable for use with implantable conduits, e.g. for cardiac vascular applications, because the configuration of the device does not permit connection to a graft conduit.

There is a need in the prior art to provide a size-variable implantable device which may vary in size, e.g. expand on contract, in response to environmental conditions, parameters, and/or stimuli, and/or in response to a clinician's command.

There is a need in the prior art to provide an expandable anastomosis device which does not require replacement caused by growth of the patient, e.g. a child.

There is a need in the prior art to provide an anastomosis device, and in particular an anastomosis device for securing a surgical conduit to a patient's anastomosis site, which is capable of expanding, for example in response to growth of the patient.

There is a need in the prior art to provide a contractible implantable device which is capable of contracting, for example in response to environmental conditions, parameters, and/or stimuli, and/or in response to a clinician's command.

It is an object of at least one embodiment of at least one aspect of the present invention to seek to obviate or at least mitigate one or more disadvantages in the prior art.

### SUMMARY OF INVENTION

According to a first aspect of the present invention there is provided a size-variable implantable device comprising:
a body comprising at least one attaching portion configured for securing the device to a surgical site of a patient;
a size-controlling member configured for controlling at least one dimension of the device; and
an actuator arranged to actuate the size-controlling member.

The surgical site of the patient may comprise an anastomosis site.

Typically, the device may comprise an opening, e.g. a substantially central opening, which may define a passageway, e.g. for a bodily fluid. In use, the device may be disposed such that the opening may be substantially aligned with a lumen, conduit, and/or orifice of a patient.

The opening may be defined by an inner portion, e.g. an inner surface, of the body and/or of the at least one attaching portion.

The opening may be substantially oval, e.g. substantially circular. An outer portion and/or periphery of the device may be substantially oval, e.g. substantially circular. By such provision the device may substantially conform to a lumen, conduit, and/or orifice of a patient.

The size-controlling member may be configured for controlling at least one dimension of the body and/or of the attaching portion.

The at least one dimension may comprise a diameter of the device, e.g. of the body and/or of the attaching portion. The at least one dimension may comprise a diameter of the opening, and/or a diameter of an outer portion or periphery of the device. The term “diameter” is herein not restricted to a device of circular configuration, but will be understood to define a dimension of the device taken across a width of the device, when placed inside a patient's lumen, conduit, and/or orifice, in use, and passing through a substantially central part of the device and/or opening thereof.

The body, e.g. the at least one attaching portion, may comprise at least one contacting portion or surface configured for contacting a surgical site of a patient.

The at least one contacting portion or surface and/or the at least one attaching portion or surface may be substantially flat, smooth, continuous and/or may be substantially free of crenellations. Such arrangement may allow expansion and/or contraction of the size-variable implantable device, while minimising the risks of potential damage to surrounding tissue, in use, by providing continuous and/or snug fit and/or contact with the tissue.

The at least one contacting portion and/or the at least one at least one attaching portion may comprise at least one flange. The contacting portion, at least one attaching portion and/or at least one flange may have a substantially uniform profile.

Alternatively, or additionally, an outer, e.g. peripheral or circumferential, portion of the attaching portion may comprise the contacting portion or surface configured for contacting a surgical site of a patient.

The at least one attaching portion may comprise at least one aperture. The at least one aperture may comprise a plurality of apertures, e.g. suture holes. Conveniently, the apertures and/or suture holes may be radially located relative to a longitudinal axis of the device. Alternatively, the at least one aperture may comprise a single aperture, e.g. extending substantially circularly within the at least one attaching portion.

In use, the device may be attached to a surgical site of a patient via the at least one attaching portion by performing interrupted suturing. The use of interrupted suturing consists of independently suturing the device at various attachment points, wherein the suturing material is cut between the various attachment points. By such provision, the suturing material does not affect the expansion or contraction of the device, such that expansion and/or contraction of the device is capable of causing expansion and/or contraction of the surgical site. In contrast, the use of continuous suturing may adversely affect the ability of the device to expand and/or contract due to the suturing material interconnecting a number of attachment points.

Alternatively, or additionally, the device may be attached to as surgical site of a patient by using a physiologically compatible adhesive, such as a cyanoacrylate-based adhesive. In such instance, the contacting portion or surface of the at least one attaching portion may be configured for receiving a physiologically compatible adhesive prior to contacting the surgical site.

The body may comprise a receiving portion configured for receiving the size-controlling member.

The receiving portion may comprise at least one retaining portion configured for contacting the size-controlling member.

The at least one retaining portion may comprise at least one annular rim or ridge.

When the implantable device is an expandable device, the size-controlling member may be provided on an inner side of the at least one retaining portion. By such provision, when the size-controlling member expands, it may contact the at least one retaining portion and may cause expansion of the device.

When the implantable device is a contractible device, the size-controlling member may be provided on an outer side of the at least one retaining portion. By such provision, when the size-controlling member contracts, it may contact the at least one retaining portion and may cause contraction expansion of the device.

The receiving portion may comprise a plurality of, e.g. a pair of, retaining portions, which may comprise a pair of annular rims defining an annular groove configured for receiving the size-controlling member. By such provision the receiving portion may be suitable for use in both an expandable and a contractible device.

The body may comprise a mounting portion for mounting one or more conduits, preferably one conduit. By such provision the implantable device may be used as part of an implantable prosthetic conduit, e.g. of an expandable conduit.

The mounting portion may protrude away, e.g. in a longitudinal direction, from the body of the device, e.g. from the at least one attaching portion. For example, the mounting portion may be substantially aligned, tangential or parallel to an axis of the opening and/or of the lumen, conduit and/or orifice.

The mounting portion may comprise an annular ridge or rim, configured for mounting a conduit on an inner and/or outer side thereof.

The mounting portion may be formed integrally with the conduit. Alternatively, the mounting portion may be formed separately from the body and/or from the attaching portion.

The conduit may be attached to the device either before or after implantation inside a patient's body.

The device may comprise the conduit.

The mounting portion may be provided near an inner portion of the body and/or of the at least one attaching portion. The mounting portion may define an inner surface defining the opening.

Alternatively, the mounting portion may be provided near a central or outer portion of the body and/or of the at least one attaching portion. The mounting portion may define an outer surface of the body.

Advantageously, the receiving portion, retaining portion and/or mounting portion may be formed integrally with the body and/or with at least one attaching portion. This may help improve the robustness and mechanical integrity of the device.

At least part of the body, e.g. the at least one attaching portion, may be made from a deformable, e.g. an expandable and/or contractible, material. The material may comprise a resilient, elastic and/or stretchable material, such as a resilient, elastic and/or stretchable polymer, e.g. silicone, Dacron®-coated silicone, or the like.

In one embodiment, the body may be made from a deformable, e.g. an expandable and/or contractible, material. The material may comprise a resilient, elastic and/or stretchable material, such as a resilient, elastic and/or stretchable polymer, e.g. silicone, Dacron®-coated silicone, or the like.

Provision of the body being made from an elastic and/or stretchable material allows expansion and/or contraction of the size-variable implantable device, while minimising the risks of potential damage to surrounding tissue, in use. Such an arrangement may also permit substantially symmetrical radial expansion and/or contraction.

The size-controlling member may comprise a substantially rigid member. The term “substantially rigid” will be herein understood to mean a member which is of higher rigidity than the body, such that expansion and/or contraction of the size-controlling member may cause expansion and/or contraction of the body, e.g. of the at least one attaching portion.

The size-controlling member may be provided within a receiving portion of the body such that expansion and/or contraction of the size-controlling member causes expansion and/or contraction of the body, e.g. of the attaching portion.

The size-controlling member may be arranged to be deployable between a contracted configuration in which the device is in a contracted configuration, and an expanded configuration in which the device is in an expanded configuration.

In use, before deployment in a patient's body, the device may be provided in a relaxed state.

When the device is an expandable device, the device in a relaxed state may be in a contracted configuration.

When the device is a contractible device, the device in a relaxed state may be in an expanded configuration.

The size-controlling member may comprise an elongate member, e.g. an elongate strip, wound radially relative to the receiving portion and/or at least one retaining portion.

The size-controlling member may comprise an elongate member arranged in a spiral-like configuration within the receiving portion. The elongate member may comprise a first or inner end near an inner end of the spiral, and a second or outer end near an outer end of the spiral.

In use, the elongate member may define at least one complete loop within the receiving portion.

In use, in a contracted configuration, the elongate member may define a plurality of overlapping loops.

In use, in an expended configuration, the elongate member may define at least one complete loop.

The device may be sized such that, in a fully expanded configuration for the intended surgical application, the elongate member may define one complete loop, plus optionally a small amount of overlap between the first and second end thereof. By such provision, the weight of the device may be minimised by minimising the amount of material required to allow deployment to and/or from full expansion.

The device may further comprise a movement restrictor configured for restricting expansion and/or contraction of the size-controlling member.

The movement restrictor may be attached, e.g. permanently attached to a portion of the size-controlling member, e.g. at or near a first end or second end thereof.

The movement restrictor may be configured to allow movement, e.g. longitudinal slidable movement, of the size-controlling member through the movement restrictor.

Advantageously, the movement restrictor may comprise a one-way movement restrictor.

The size-controlling member may comprise restricting features capable engaging with the movement restrictor.

In one embodiment, the size-controlling member may comprise grooves and/or ridges arranged, e.g. transversely, relative to a length of the size-controlling member, e.g. of the elongate member. By such provision the mechanism of the movement restrictor upon the size-controlling member may be likened to a “Jubilee™ clip”, configured either for contracting or for expanding.

The size-controlling member may be made of a relatively strong and/or robust material such as metal, e.g. stainless steel or titanium, or such as plastic, e.g. polypropylene, polycarbonate, polyurethane, polyacrylates, or the like.

The size-controlling member may have a thickness of 0.05-1 mm, typically about 0.2 mm.

The size-controlling member may have a width of 8-16 mm, typically about 12 mm.

The size-controlling member may have a length of 5-20 cm, typically about 10 cm. The skilled person will appreciate that the length of the size-controlling member may depend upon the intended application and deployment site. In one embodiment, for use of the device as an expandable anastomosis device, the length of the size-controlling member may be such that the diameter of the size-controlling member may vary between approximately 1 cm in diameter in a contracted configuration and approximately 3 cm in diameter in an expanded configuration.

Preferably, the actuator arranged to actuate the size-controlling device may be motorised and/or may comprise a motor.

The motor may comprise a micromotor, e.g. an ultrasonic motor or a piezoelectric motor. Advantageously, the use of an ultrasonic motor may generate more mechanical power than a conventional micromotor.

Advantageously, the actuator may be arranged to expand and/or contract the size-controlling device at a predetermined rate and/or at a constant rate. By such provision, in-situ actuation may be performed accurately and reliably. This also allows expansion and/or contraction of the device at a very slow rate, e.g. over a number of days, weeks, or months, which may help minimise or avoid damage to the tissue at or near the surgical or anastomosis site.

The actuator may comprise a housing configured for receiving the motor.

The actuator may comprise the movement restrictor. This allows reliable actuation and movement restriction of the size-controlling member using a simple, compact and light-weight construction.

Conveniently, the actuator may be remotely accessible or operable, e.g. by remote communication means, preferably wireless and/or electromagnetic communication means, such as Bluetooth™ technology, WiFi technology and the like.

The device may comprise one or more sensors for detecting, measuring, collecting, storing, and/or communicating data.

The sensor(s) may be adapted to measure environmental data, information or parameters, e.g. physiological data, particularly in the region of deployment of the device within a patient's body.

In one embodiment, the sensor(s) may be adapted to measure blood flow and/or pressure at or near the deployment site, e.g. at or near the anastomosis site. Such a sensor may be particularly useful for use when the implantable device is an anastomosis device, e.g. for use in cardiac procedures. By such provision, the sensor may be capable of measuring, storing and/or transmitting data useful in determining whether or not the size of the device is of appropriate size to ensure adequate blood flow through the device.

In another embodiment, the sensor(s) may be adapted to measure acidity at or near the surgical site. Such a sensor may be particularly useful for use when the implantable device is a device for use to control gastric reflux.

Conveniently, the data measured and/or collected by the sensor may be remotely accessible and/or retrievable, e.g. by remote communication means, preferably wireless and/or electromagnetic communication means, such as Bluetooth™ technology, WiFi technology and the like.

The sensor may automatically cause the actuator to actuate the size-controlling device when one or more of the detected parameters reach a predetermined value. For example, when the device is an expandable anastomosis device for cardiac applications, the actuator may be automatically activated when the sensor measures blood flow and/or pressure values above normal levels.

Alternatively, the actuator may be remotely accessible or operable by a clinician, for example based on the data collected and communicated by the sensor at the anastomosis site.

The actuator, motor, and/or sensor may be powered by a power supply.

The power supply may comprise one or more batteries. The battery(ies) may be rechargeable by transcutaneous coil charging.

The power supply may comprise a biogenerated power supply, e.g. may use body chemistry of the patient as a power supply.

Conveniently, the power supply may comprise one or more piezoelectric or electrokinetic patches.

According to a second aspect of the present invention there is provided a size-variable implantable device comprising:
a body comprising at least one attaching portion configured for securing the device to a surgical site of a patient;
a size-controlling member configured for controlling at least one dimension of the device;
an actuator arranged to actuate the size-controlling member; and
a mounting portion for mounting and/or connecting one or more conduits, e.g. one conduit, to the device.

The size-variable implantable device may comprise an expandable implantable device.

Such an implantable device may be used as part of an implantable prosthetic conduit, e.g. of an expandable conduit.

The body may comprise and/or may be provided with the mounting portion.

The mounting portion may be formed integrally with the conduit. Alternatively, the mounting portion may be formed separately from the body and/or from the attaching portion.

The conduit may be attached to the device either before or after implantation inside a patient's body.

The mounting portion may protrude away from the body, e.g. from the at least one attaching portion. For example, the mounting portion may be substantially aligned, tangential or parallel to an axis of the opening and/or of the lumen, conduit and/or orifice.

The mounting portion may comprise an annular ridge or rim, configured for mounting a conduit on an inner and/or outer side thereof.

The surgical site of the patient may comprise an anastomosis site.

The features described above in respect of the size-variable implantable device according to the first aspect of the invention may apply to the expandable implantable device according to the second aspect of the invention, and are not repeated here for brevity.

According to a third aspect of the present invention there is provided a prosthetic conduit comprising:
at least one size-variable implantable device comprising:
   a body comprising at least one attaching portion configured for securing the device to a surgical site of a patient;
   a size-controlling member configured for controlling at least one dimension of the device; and
   an actuator arranged to actuate the size-controlling member; and
   at least one implantable conduit secured to the at least one size-variable implantable device.

The body may comprise a mounting portion for mounting the at least one implantable conduit.

The mounting portion may protrude away from the body, e.g. from the at least one attaching portion. For example, the mounting portion may be substantially aligned, tangential or parallel to an axis of the opening and/or of the lumen, conduit and/or orifice.

The mounting portion may comprise an annular ridge or rim, configured for mounting a conduit on an inner and/or outer side thereof.

The mounting portion may be formed integrally with the conduit. Alternatively, the mounting portion may be formed separately from the body and/or from the attaching portion.

The conduit may be attached to the device either before or after implantation inside a patient's body.

The surgical site of the patient may comprise an anastomosis site.

The prosthetic conduit may comprise two size-variable implantable devices, one size-variable implantable device being provided at each end of the implantable conduit.

The implantable conduit may comprise an expandable conduit.

The expandable conduit may comprise a balloon-expandable conduit. By such provision, after expansion of the size-variable implantable device, the expandable conduit attached thereto may be expanded via a relatively minor and safe routine procedure comprising balloon expansion.

Preferably, the at least one size-variable implantable device may comprise an expandable implantable device.

The features described above in respect of the size-variable implantable device according to the first aspect or second aspect of the invention may apply to the prosthetic conduit according to the third aspect of the invention, and are not repeated here for brevity.

According to a fourth aspect of the present invention there is provided a size-variable implantable device comprising:
a body comprising at least one attaching portion configured for securing the device to a surgical site of a patient;
a size-controlling member configured for controlling at least one dimension of the device; and
an actuator arranged to actuate the size-controlling member,
wherein the body is made of an elastic and/or stretchable material.

Provision of the body being made from an elastic and/or stretchable material allows expansion and/or contraction of the size-variable implantable device, while minimising the risks of potential damage to surrounding tissue, in use. Such an arrangement may also permit substantially radially symmetrical expansion and/or contraction.

The body may be made from a resilient, elastic and/or stretchable polymer such as silicone, Dacron®-coated silicone, or the like.

The body, e.g. the at least one attaching portion, may comprise at least one contacting portion or surface configured for contacting a surgical site of a patient.

The at least one contacting portion or surface and/or the at least one attaching portion or surface may be substantially flat, smooth, continuous and/or may be substantially free of crenellations. Such arrangement may allow expansion and/or contraction of the size-variable implantable device, while minimising the risks of potential damage to surrounding tissue, in use, by providing a continuous and/or snug fit and/or contact with the tissue.

The at least one contacting portion and/or the at least one at least one attaching portion may comprise or may be formed as a flange. The contacting portion, at least one attaching portion and/or flange may have a substantially uniform profile.

The surgical site of the patient may comprise an anastomosis site.

The features described above in respect of the size-variable implantable device according to the first aspect or second aspect of the invention may apply to the size-variable implantable device according to the fourth aspect of the invention, and are not repeated here for brevity.

According to a fifth aspect of the present invention there is provided a size-variable implantable device comprising:
a body comprising at least one attaching portion configured for securing the device to a surgical site of a patient;
a size-controlling member configured for controlling at least one dimension of the device;
an actuator arranged to actuate the size-controlling member; and
one or more sensors for detecting, measuring, collecting, storing, and/or communicating data.

The sensor(s) may be adapted to measure environmental data, information or parameters, e.g. physiological data, particularly in the region of deployment of the device within a patient's body.

In one embodiment, the sensor(s) may be adapted to measure blood flow and/or pressure at or near the deployment site, e.g. at or near the anastomosis site. Such a sensor may be particularly useful for use when the implantable device is an anastomosis device, e.g. for use in cardiac procedures. By such provision, the sensor may be capable of measuring, storing and/or transmitting data useful in determining whether or not the size of the device is of appropriate size to ensure adequate blood flow through the device.

In another embodiment, the sensor(s) may be adapted to measure acidity at or near the surgical site. Such a sensor may be particularly useful for use when the implantable device is a device for use to control gastric reflux.

Conveniently, the data measured and/or collected by the sensor may be remotely accessible and/or retrievable, e.g. by remote communication means, preferably wireless and/or electromagnetic communication means, such as Bluetooth™ technology, WiFi technology and the like.

Provision of sensors within or integrated with the size-variable implantable device may avoid the need for separate diagnostic procedures in order to assess the condition of, and/or or obtain data from, the surgical site.

The sensor may automatically cause the actuator to actuate the size-controlling device when one or more of the detected parameters reach a predetermined value. For example, when the device is an expandable anastomosis device for cardiac applications, the actuator may be automatically activated when the sensor measures blood flow and/or pressure values above normal levels.

Alternatively, the actuator may be remotely accessible or operable by a clinician, for example based on the data collected and communicated by the sensor at the anastomosis site.

The actuator, motor, and/or sensor may be powered by a power supply.

The power supply may comprise one or more batteries. The battery(ies) may be rechargeable by transcutaneous coil charging.

The power supply may comprise a biogenerated power supply, e.g. may use body chemistry of the patient as a power supply.

Conveniently, the power supply may comprise one or more piezoelectric or electrokinetic patches.

The features described above in respect of the size-variable implantable device according to the first, second or fourth aspect of the invention may apply to the size-variable implantable device according to the fifth aspect of the invention, and are not repeated here for brevity.

According to a sixth aspect of the present invention there is provided a method of performing surgery on a patient, comprising attaching a size-variable implantable device to a surgical site of a patient, the device comprising:
a body comprising at least one attaching portion configured for securing the device to a surgical site of a patient;
a size-controlling member configured for controlling at least one dimension of the device; and
an actuator arranged to actuate the size-controlling member.

The surgical site of the patient may comprise an anastomosis site.

The method may comprise attaching the size-variable implantable device to a surgical site of a patient by performing interrupted suturing through at least one aperture of the attaching portion. By such provision, the suturing material does not affect the expansion or contraction of the device, such that expansion and/or contraction of the device is capable of causing expansion and/or contraction of the surgical site.

The method may comprise expanding the anastomosis site by expanding the size-variable implantable device.

The method may comprise contracting the surgical site by contracting the size-variable implantable device.

The method may comprise expanding and/or contracting the size-variable implantable device remotely, e.g. by using by remote communication means preferably wireless and/or electromagnetic communication means, such as Bluetooth™ technology, WiFi technology and the like.

By such provision the method does not require replacement of the size-variable implantable device caused by growth of the patient, e.g. a child.

The method may comprise measuring, transmitting, recording, and/or considering data obtained by sensor(s) of the size variable implantable device, and adapted to measure environmental data, information or parameters, e.g. physiological data, particularly in the region of deployment of the device within a patient's body.

The method may comprise expanding an implantable conduit attached to the device, e.g. a balloon-expandable conduit.

The method may comprise attaching a size-variable implantable device according to the first, second, fourth or fifth aspect of the invention, or a prosthetic conduit according to the third aspect of the present invention, and features associated therewith.

### BRIEF DESCRIPTION OF DRAWINGS

Embodiments of the invention will now be given by way of example only, and with reference to the accompanying drawings, which are:

FIG. 1 a perspective view of as size-variable device according to a first embodiment of the present invention;

FIG. 2 a cross sectional view of the size-variable device of FIG. 1;

FIG. 3 a perspective view of the size-controlling member and actuator of the device of FIG. 1;

FIG. 4 a perspective view of the size-controlling member of the device of FIG. 1;

FIG. 5a an elevated front view of the size-controlling member and actuator of the device of FIG. 1, in a contracted configuration;

FIG. 5b an elevated front view of the size-controlling member and actuator of the device of FIG. 1, in an expanded configuration;

FIG. 6a a perspective view of a prosthetic conduit according to a second embodiment of the present invention;

FIG. 6b a perspective view of the prosthetic conduit of FIG. 6a, after expansion of the expandable implantable devices;

FIG. 6c a perspective view of the prosthetic conduit of FIG. 6a, after expansion of the implantable conduit.

### DETAILED DESCRIPTION OF DRAWINGS

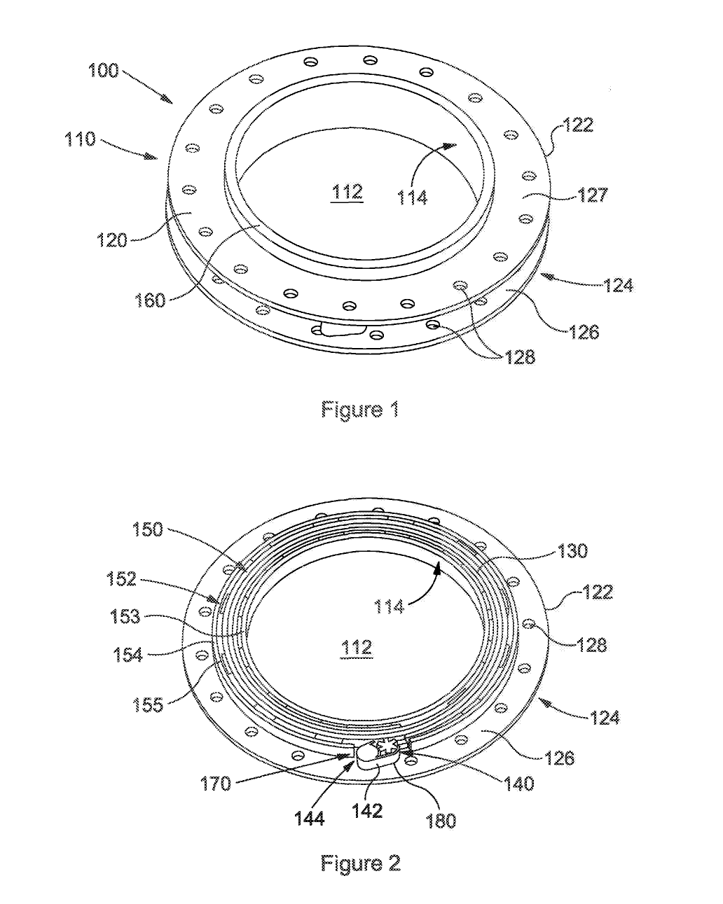

Referring to FIGS. 1 to 5b, there is shown a size-variable implantable device, generally designated 101, according to first embodiment of the present invention.

The size-variable implantable device 101 comprises a body 110 comprising at least one attaching portion 120 configured for securing the device to a surgical site of a patient; a size-controlling member 130 configured for controlling at least one dimension of the device; and an actuator 140 arranged to actuate the size-controlling member 130.

In this embodiment the size-variable implantable device 101 is an expandable implantable device for securing a surgical conduit to a patient's anastomosis site.

The device 101 comprises a central opening 112 defining a passageway for a bodily fluid such as blood. In use, the device 101 is disposed such that the opening 112 is substantially aligned with a lumen of a vascular pathway.

The opening 112 is defined by an inner surface 114 of the body 110, and is substantially circular. In other embodiments, the opening 112 may be substantially oval, or may be of any other suitable shape to conform to the shape of a lumen, conduit, and/or orifice of a patient where the device is to be implanted.

The size-controlling member 130 is configured for controlling the diameter of the body 110, and in particular the diameter of the attaching portion 120 such as an outer periphery or circumference 122 thereof.

The attaching portion 120 comprises flanges 126,127 which have a substantially uniform profile.

With reference to FIG. 2, the attaching portion 120 comprises a contacting surface 124 configured for contacting a surgical site of a patient. The contacting surface 124 is substantially flat, smooth, continuous and is substantially free of crenellations. Such arrangement allows expansion and/or contraction of the size-variable implantable device, while minimising the risks of potential damage to surrounding tissue, in use. Alternatively, or additionally, the outer periphery or circumference 122 of the attaching portion 120 or flanges 126,127 comprises the contacting surface 124 configured for contacting a surgical site of a patient.

The attaching portion 120 comprises a plurality of radially disposed apertures 128 for use as suture holes. In an alternative embodiment, the apertures 128 may be replaced by a single aperture extending substantially circularly within the attaching portion 120.

In use, the device 101 is attached to a surgical site of a patient via the attaching portion 120 by performing interrupted suturing using the apertures 128. The use of interrupted suturing consists of independently suturing the device 101 at various attachment points 128, wherein the suturing material is cut between the various attachment points 128. By such provision, the suturing material does not affect the expansion or contraction of the device 101, such that expansion and/or contraction of the device 101 is capable of causing expansion and/or contraction of the surgical site. In contrast, the use of continuous suturing may adversely affect the ability of the device to expand and/or contract due to the suturing material interconnecting a number of attachment points.

Alternatively, or additionally, the device 101 may be attached to a surgical site of a patient by using a physiologically compatible adhesive, such as a cyanoacrylate-based adhesive. In such instance, the contacting surface 124 of the attaching portion 120 may be configured for receiving a physiologically compatible adhesive prior to contacting the surgical site.

With reference to FIG. 2, the body 110 comprises a receiving portion 150 configured for receiving the size-controlling member 130.

The receiving portion 150 comprises a retaining portion 152 configured for contacting the size-controlling member 130.

In this embodiment, the retaining portion 152 comprises a pair of annular rims comprising an inner annular rim 153 and an outer annular rim 154. The size-controlling member 130 is located within an annular groove 155 between the inner annular rim 153 and an outer annular rim 154.

The size-controlling member 130 is on an inner side of the outer annular rim 154. By such provision, when the size-controlling member 130 expands, it will contact the outer annular rim 154 to cause expansion of the device 101. The size-controlling member 130 is boated on an outer side of the inner annular rim 153. By such provision, when the size-controlling member 130 contracts, it will contact the inner annular rim 153 to cause contraction of the device 101.

The body 110 comprises a front flange 127 and a rear flange 126, the size-controlling member 130 being securely provided within the retaining portion 152 between the front flange 127 and a rear flange 126.

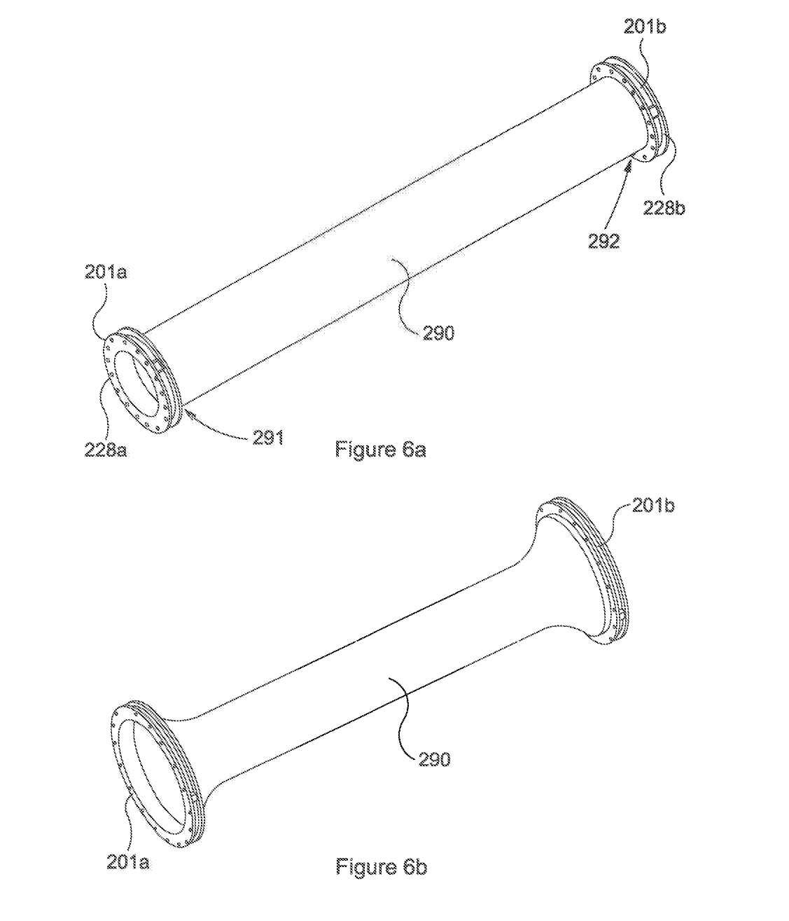

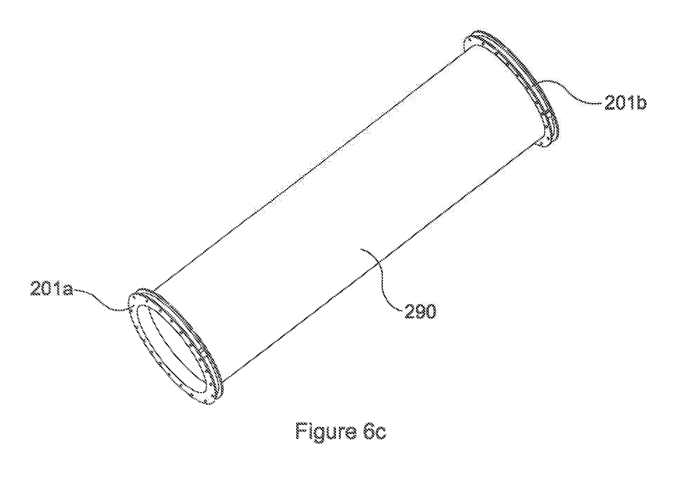

The body 110 comprises a mounting portion 160 for mounting one or more conduits, preferably one conduit. By such provision the implantable device 101 may be used as part of an implantable prosthetic conduit, e.g. of an expandable conduit, as shown in FIGS. 6a to 6c.

As shown in FIG. 1, the mounting portion 160 is formed as an annular rim which protrudes away from the attaching portion 120 of the body 110, and is substantially aligned, tangential or parallel to an axis of the opening and/or of the lumen, conduit and/or orifice.

Conveniently, the mounting portion 160 is formed integrally with the body 110 including the attaching portion 120. By such provision, a conduit may be attached to the device either before or after implantation inside a patient's body.

In this embodiment, the mounting portion 160 is provided near an inner portion of the body 110. The mounting portion 160 thus defines an inner surface 114 defining the opening 112.

In an alternative embodiment, the mounting portion may be provided near a central or outer portion of the body 110.

Advantageously, the receiving portion 150, retaining portion 152, mounting portion 160, and attaching portion 120 are formed integrally with the body 110, which helps improve the robustness and mechanical integrity of the device 101.

The body 110 is made from a deformable, e.g. expandable and/or contractible material. The material comprises a resilient, elastic and/or stretchable material, particularly a resilient, elastic and/or stretchable polymer such as silicone, Dacron®-coated silicone, or the like. Provision of the body 110 being made from an elastic and/or stretchable material allows expansion and/or contraction of the device 101, while minimising the risks of potential damage to surrounding tissue, in use. Such an arrangement may also permit substantially radially symmetrical expansion and/or contraction of the body 110.

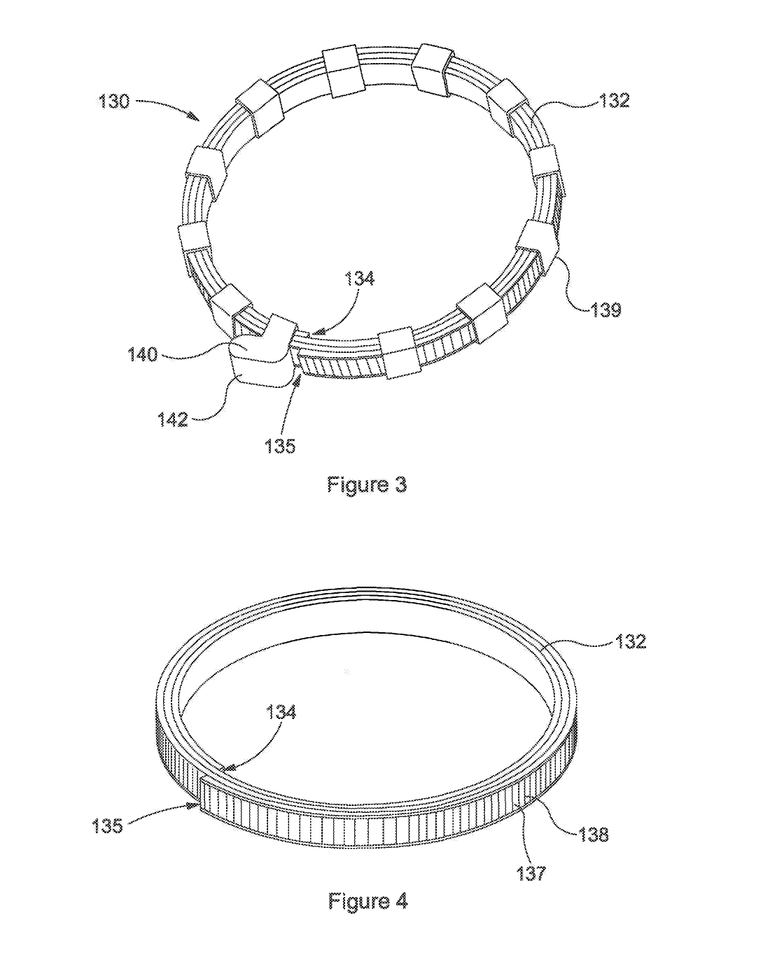

Referring to FIGS. 3, 4, 5a and 5b, the size-controlling member 130 comprises a substantially rigid member 132. The term “substantially rigid” will be herein understood to mean a member 130 which is of higher rigidity than the body 110, such that expansion and/or contraction of the size-controlling member 130 may cause expansion and/or contraction of the body 110, particularly of the at least one attaching portion 120.

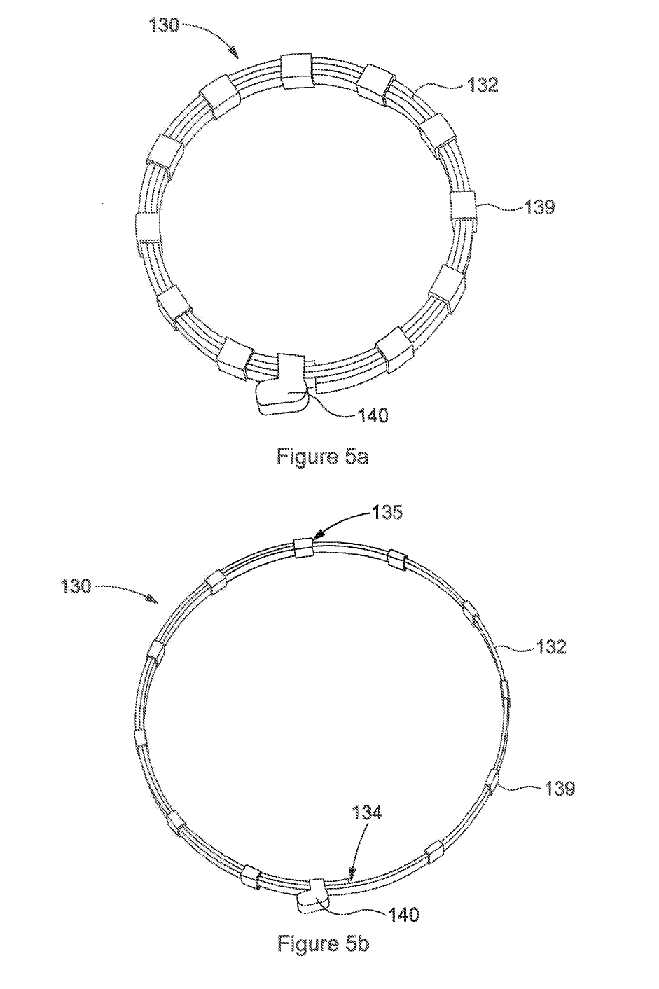

The size-controlling member 130 is arranged to be deployable between a contracted configuration in which the size-controlling member 130 is in a contracted configuration, as shown in FIG. 5a, and an expanded configuration in which the size-controlling member 130 is in an expanded configuration, as shown in FIG. 5b.

The size-controlling member 130 comprises a radially wound elongate strip 132. The elongate strip 132 is arranged in a spiral-like configuration within the receiving portion 150. The elongate strip 132 comprises a first or inner end 134 near an inner end of the spiral, and a second or outer end 135 near an outer end of the spiral.

In use, the elongate strip 132 defines at least one compete loop within the receiving portion.

In use, in the contracted configuration shown in FIG. 5a, the elongate strip 132 defines a plurality of overlapping loops. In the expanded configuration shown in FIG. 5b, the elongate strip 132 defines one complete loop, plus a small amount of overlap between the inner end 134 and the outer end 135 thereof. By such provision, the weight of the device 101 may be minimised by minimising the amount of material required to allow deployment to and/or from full expansion.

The size-controlling member 130 comprises a plurality of guides 139 arranged to maintain the alignment and/or spiral arrangement of the elongate strip 132.

The device 101 further comprises a movement restrictor 180 configured for restricting expansion and/or contraction of the size-controlling member 130. The movement restrictor 180 is configured to allow longitudinal slidable movement of the size-controlling member 130 through the movement restrictor 180.

Advantageously, the movement restrictor 180 comprises a one-way movement restrictor. The size-controlling member 130 comprises restricting features 136 capable of engaging with the movement restrictor. In this embodiment, the size-controlling member 130 comprises grooves 137 and ridges 138 arranged transversely relative to a length of the elongate strip 132. By such provision the mechanism of the movement restrictor 180 upon the size-controlling member 130 may be likened to a “Jubilee™ clip”.

The elongate strip 132 is made of metal such as stainless steel or titanium, so as to provide the desired strength and robustness.

The elongate strip 132 has a thickness of 0.05-1 mm, typically about 0.2 mm.

The size elongate strip 132 has a width of 8-16 mm, typically about 12 mm.

The elongate strip 132 has a length of 5-20 cm, typically about 10 cm. The skilled person will appreciate that the length of the elongate strip 132 depends upon the intended application and deployment site. in one embodiment, for use of the device as an expandable anastomosis device, the length of the elongate strip 132 may be such that the diameter of the elongate strip 132 may vary between approximately 1 cm in diameter in a contracted configuration and approximately 3 cm in diameter in an expanded configuration.

In this embodiment, the actuator 140 arranged to actuate the size-controlling device 130 is motorised, e.g. by use of a micromotor such as an ultrasonic motor.

The actuator comprises a housing 142 configured for receiving the motor 144.

The actuator also comprises the movement restrictor 180. This allows reliable actuation and movement restriction of the size-controlling member 130 using a simple, compact and light-weight construction.

Conveniently, the actuator 140 can be remotely accessible or operable, e.g. by remote communication means, preferably wireless and/or electromagnetic communication means, such as Bluetooth™ technology, WiFi technology and the like.

The device comprises one or more sensors 170 for detecting, measuring, collecting, storing, and/or communicating data.

The sensor(s) 170 are adapted to measure environmental data, information or parameters, e.g. physiological data, particularly in the region of deployment of the device within a patient's body. In this embodiment, the sensor(s) 170 are adapted to measure blood flow and blood pressure at the anastomosis site. Such a sensor may be particularly useful for use when the implantable device is an anastomosis device, e.g. for use in cardiac procedures. By such provision, the sensor 170 is capable of measuring, storing and/or transmitting data useful in determining whether or not the size of the device 101 is of appropriate size to ensure adequate blood flow through the device 101.

In alternative embodiments, the sensor(s) 170 may be adapted to measure acidity at or near the surgical site. Such a sensor may be particularly useful for use when the implantable device is a device for use to control gastric reflux.

Conveniently, the data measured and/or collected by the sensor can be remotely accessed and/or retrieved by remote communication means, preferably wireless and/or electromagnetic communication means, such as Bluetooth™ technology, WiFi technology and the like.

Advantageously, the actuator 140 and/or movement restrictor 180 may be remotely accessible or operable by a clinician, for example based on the data collected and communicated by the sensor at the anastomosis site.

Alternatively, the sensor 170 may automatically cause the actuator 140 and/or movement restrictor 180 to actuate the size-controlling device 130 when one or more of the detected parameters reach a predetermined value. For example, when the device 110 is an expandable anastomosis device for cardiac applications, the actuator 140 may be automatically activated when the sensor 170 measures blood flow and/or pressure values above normal levels.

The actuator 140, motor 144, and/or sensor 170 are powered by a power supply (now shown). The power supply may comprise one or more batteries such as battery(ies) may be rechargeable by transcutaneous coil charging. The power supply may comprise a biogenerated power supply, e.g. may use body chemistry of the patient as a power supply. Conveniently, the power supply may comprise one or more piezoelectric or electrokinetic patches.

Referring to FIGS. 6a to 6c, there is shown a perspective view of a prosthetic conduit, generally designated 200, according to a second embodiment of the present invention. The prosthetic conduit 200 comprises an implantable conduit 290, and a pair of size-variable implantable devices 201a,201b. The size-variable implantable devices 201a,201b are generally similar to the size-variable implantable device 101 of FIGS. 1 to 5b, like parts being denoted by like numerals, incremented by “100”. The size-variable implantable devices 201a,201b are secured at both ends 291,292 of the implantable conduit 290 via their respective mounting portions 260a,260b.

In this embodiment, the implantable conduit 290 is a balloon-expandable conduit, and the size-variable implantable devices 201a,201b are expandable implantable devices 201a,201b.

FIG. 6a shows the prosthetic conduit before implantation, in a non-expanded configuration.

In use, the prosthetic conduit 200 is provided at an anastomosis site, e.g. within a vascular pathway, and the implantable devices 201a,201b are secured to a surgical site of a patient by suturing the devices 201a,201b via suture holes 228a,228b.

When the prosthetic conduit 200 requires to be expanded, for example due to growth of a patient, the implantable devices 201a,201b are expanded, as shown in FIG. 6b. Expansion of the implantable devices 201a,201b is carried out as described in respect of FIGS. 1 to 5b.

Once the implantable devices 201a,201b have been expanded to a desired dimension, the implantable conduit 290 is expanded to a desired dimension by balloon expansion in situ. The prosthetic conduit 200 is then in an expanded configuration, as shown in FIG. 6c.

If further expansion is required at a later stage, the same procedure may be repeated as many times as required.

It will be appreciated that the embodiments of the invention hereinbefore described are given by way of example only and are not meant to limit the scope thereof in any way. For example, while the dimensions of the various embodiments have been provided based on a human patient of average size, the skilled person would appreciate that smaller or larger devices may be fabricated for alternative applications, e.g. for animal surgery. Thus, larger devices would be required for performing surgery on e.g. horses, while smaller devices would be required for performing surgery on e.g. dogs or cats.

1. A size-variable implantable device comprising:
a body comprising at least one attaching portion configured for securing the device to a surgical site of a patient;
a size-controlling member configured for controlling at least one dimension of the device;
an actuator arranged to actuate the size-controlling device; and
a mounting portion for mounting and/or connecting at least one conduit to the device.

2. A size-variable implantable device according to claim 1, wherein the body comprises the mounting portion.

3. A size-variable implantable device according to claim 1, wherein the mounting portion is formed integrally with the body.

4. A size-variable implantable device according to claim 1, wherein the device comprises an opening defining a fluid passageway.

5. A size-variable implantable device according to claim 4, wherein the opening is defined by an inner surface of the body and/or of the at least one attaching portion.

6. A size-variable implantable device according to claim 1, wherein the mounting portion protrudes away from the body, and is substantially aligned, tangential or parallel to an axis of the opening.

7. A size-variable implantable device according to claim 1, wherein the size-controlling member is configured for controlling at least one dimension of the body and/or of the attaching portion.

8. A size-variable implantable device according to claim 1, wherein the at least one attaching portion comprises a plurality of aperture for securing the device to a surgical site of a patient.

9. A size-variable implantable device according to claim 1, wherein the at least one attaching portion comprises at least one contacting portion or surface configured for contacting a surgical site of a patient.

10. A size-variable implantable device according to claim 9, wherein the at least one contacting portion or surface is substantially flat, smooth, continuous and/or is substantially free of crenellations.

11. A size-variable implantable device according to claim 1, wherein the body comprises a receiving portion configured for receiving the size-controlling member.

12. A size-variable implantable device according to claim 11, wherein the receiving portion comprises at least one retaining portion configured for contacting the size-controlling member, the retaining portion comprising at least one annular rim or ridge.

13. A size-variable implantable device according to claim 1, wherein the body is made from a resilient, elastic and/or stretchable material.

14. A size-variable implantable device according to claim 13, wherein the material comprises a silicone material.

15. A size-variable implantable device according to claim 1, wherein the size-controlling member comprises a radially wound elongate strip.

16. A size-variable implantable device according to claim 1, further comprising a movement restrictor configured for restricting expansion and/or contraction of the size-controlling member.

17. A size-variable implantable device according to claim 16, wherein the size-controlling member comprises restricting features arranged for engaging with the movement restrictor.

18. A size-variable implantable device according to claim 1, wherein the size-controlling member is made of stainless steel or titanium.

19. A size-variable implantable device according to claim 1, wherein the actuator arranged to actuate the size-controlling device comprises a motorised actuator.

20. A size-variable implantable device according to claim 1, wherein the actuator is remotely accessible or operable by remote communication means.

21. A size-variable implantable device according to claim 1, wherein the device comprises one or more sensors for measuring, collecting, storing, and/or communicating data at or near the surgical site.

22. A size-variable implantable device according to claim 21, wherein the sensor(s) is adapted to measure blood flow and/or pressure at or near the surgical site.

23. A size-variable implantable device according to claim 1, wherein the size-variable implantable device is an expandable implantable device.

24. A prosthetic conduit comprising size-variable implantable device comprising:
an implantable conduit; and
at least one size-variable implantable device according to claim 1, the at least one size-variable implantable device being secured to the implantable conduit.

25. A size-variable implantable device comprising:
a body comprising at least one attaching portion configured for securing the device to a surgical site of a patient;
a size-controlling member configured for controlling at least one dimension of the device; and
an actuator arranged to actuate the size-controlling device,
wherein the body is made of an elastic and/or stretchable material.

26. A size-variable implantable device according to claim 25, wherein the body is made of an elastic and/or stretchable polymer.

27. A size-variable implantable device according to claim 26, wherein the polymer comprises a silicone material.

28. A size-variable implantable device comprising:
a body comprising at least one attaching portion configured for securing the device to a surgical site of a patient;
a size-controlling member configured for controlling at least one dimension of the device;
an actuator arranged to actuate the size-controlling device; and
one or more sensors for measuring, collecting, storing, and/or communicating data at or near the surgical site.

29. A size-variable implantable device according to claim 28, wherein the sensor(s) is adapted to measure blood flow and/or pressure at or near the surgical site.

30. A method of performing surgery on a patient, comprising attaching a size-variable implantable device to a surgical site of a patient, the device comprising:
a body comprising at least one attaching portion configured for securing the device to a surgical site of a patient;
a size-controlling member configured for controlling at least one dimension of the device; and
an actuator arranged to actuate the size-controlling device.

31-35. (canceled)
